(19) United States
(12) Patent Application Publication
(10) Pub. No.: US 2026/0041799 A1
(43) Pub. Date: Feb. 12, 2026

(54) HYDROLYSIS-RESISTANT HYDROGELS AND METHODS OF TREATMENT USING SAME

(71) Applicant: Boston Scientific Scimed, Inc., Maple Grove, MN (US)

(72) Inventors: Yen-Hao Hsu, Shrewsbury, MA (US); Heather Cirka, Hudson, MA (US); Nishant Khattar, White Bear Township, MN (US)

(73) Assignee: Boston Scientific Scimed, Inc., Maple Grove, MN (US)

(21) Appl. No.: 19/296,572

(22) Filed: Aug. 11, 2025

Related U.S. Application Data
(60) Provisional application No. 63/682,189, filed on Aug. 12, 2024.

(51) Int. Cl. A61K 49/04

(52) U.S. Cl. CPC A61K 49/0442; A61K 49/0457; A61K 2123/00

(57) ABSTRACT

In some aspects, systems for forming hydrogels are provided that comprises (i) a reactive multi-arm polymer that comprises three or more polymer arms linked to a core region, at least three of the polymer arms comprising a reactive moiety comprising a cyclic-imidyl-oxycarbonyl- end group and (ii) a polyamino compound comprising at least two amino (—NH₂) groups, wherein the reactive multi-arm polymer and the polyamino compound react to form crosslinked hydrogels that do not contain ester groups and have long-term stability in vivo. In other aspects, the present disclosure pertains to crosslinked hydrogels formed from such systems and methods of treatment using such systems.

### CROSS-REFERENCE TO RELATED APPLICATIONS

This application claims the benefit of U.S. Provisional Patent Application Ser. No. 63/682,189 filed on Aug. 12, 2024, the disclosure of which is incorporated herein by reference.

### FIELD

The present disclosure relates to hydrolysis-resistant hydrogels, to crosslinkable systems for forming such hydrolysis-resistant hydrogels, and to methods of treatment using such hydrolysis-resistant hydrogels.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion of the succinimidyl glutarate end groups have been replaced with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups of SpaceOAR® and SpaceOAR Vue® will rapidly react with the trilysine crosslinker in vivo to form a hydrogel. The hydrogel breaks down in-vivo over the course of about 6-9 months. The breakdown occurs primarily through the hydrolysis of the ester linkages in the glutarate groups.

The present disclosure provides implantable hydrogel alternatives to SpaceOAR® and SpaceOAR Vue®, which are hydrolysis-resistant and have long-term stability, thereby expanding the range of medical applications for the hydrogels.

### SUMMARY

In some aspects, a system for forming a hydrogel is provided that comprises (i) a reactive multi-arm polymer that comprises three or more polymer arms linked to a core region, at least three of the polymer arms comprising a hydrophilic polymer segment and a reactive moiety comprising a cyclic-imidyl-oxycarbonyl-C₂-C₈-alkylene-carbonylamino- end group and (ii) a polyamino compound comprising at least two amino (—NH₂) groups, wherein the reactive multi-arm polymer and the polyamino compound react to form a crosslinked hydrogel that does not contain ester groups and has long-term stability in vivo.

In some embodiments, the hydrophilic polymer segment is selected from polyalkylene oxide segments, polyester segments, polyoxazoline segments, polydioxanone segments, polypeptide segments, polyacrylamide segments, and poly(2-hydroxyethyl methacrylate) (PolyHEMA) segments.

In some embodiments, which can be used in conjunction with the above aspects and embodiments, the reactive moiety comprises a cyclic-imidyl-oxycarbonyl-1,2-dimethylene-carbonylamino- end group, a cyclic-imidyl-oxycarbonyl-1,3-trimethylene-carbonylamino- end group, a cyclic-imidyl-oxycarbonyl-1,4-tetramethylene-carbonylamino- end group, a cyclic-imidyl-oxycarbonyl-1,5-pentamethylene-carbonylamino- end group, or a cyclic-imidyl-oxycarbonyl-1,6-hexamethylene-carbonylamino- end group.

In some embodiments, which can be used in conjunction with the above aspects and embodiments, the reactive multi-arm polymer further comprises at least one iodinated aromatic group.

In some embodiments, which can be used in conjunction with the above aspects and embodiments, the core region comprises an iodinated aromatic group or wherein the —C₂-C₈-alkylene- group is substituted with an iodinated aromatic group.

In some embodiments, which can be used in conjunction with the above aspects and embodiments, the reactive multi-arm polymer further comprises at least on polymer arm that comprises at least one iodinated aromatic end group.

In some embodiments, which can be used in conjunction with the above aspects and embodiments, the reactive multi-arm polymer is formed by a process that comprises (a) reacting a multi-arm polymer that comprises three or more precursor polymer arms linked to the core region, at least three of the precursor polymer arms comprising the hydrophilic polymer segment and an amino end group, with a cyclic anhydride having a ring that comprises the —C₂-C₈-alkylene- group, and (b) reacting the reaction product of step (a) with an N-hydroxy cyclic imide compound in an ester coupling reaction to form the cyclic-imidyl-oxycarbonyl-C₂-C₈-alkylene-carbonylamino- end group.

In some aspects, the present disclosure pertains to a system for forming a hydrogel that comprises (i) a reactive multi-arm polymer that comprises three or more polymer arms linked to a core region, at least three of the polymer arms comprising a polyoxazoline segment and a reactive moiety comprising a cyclic-imidyl-oxycarbonyl- end group and (ii) a polyamino compound comprising at least two amino (—NH₂) groups, wherein the reactive multi-arm polymer and the polyamino compound react to form a crosslinked hydrogel that does not contain ester groups and has long-term stability in vivo.

In some embodiments, the polyoxazoline is a poly(2-C₁-C₆-alkyl-2-oxazoline) and wherein the reactive moiety comprises a cyclic-imidyl-oxycarbonyl-N—C₂-C₇-alkanoyl-methylamino- end group.

In some embodiments, which can be used in conjunction with the above aspects and embodiments, the polyoxazoline segment is selected from a poly(2-methyl-2-oxazoline) segment, a poly(2-ethyl-2-oxazoline) segment, a poly(2-n-propyl-2-oxazoline) segment, a poly(2-isopropyl-2-oxazoline) segment, a poly(2-n-butyl-2-oxazoline) segment, and a poly(2-isobutyl-2-oxazoline) segment.

In some embodiments, which can be used in conjunction with the above aspects and embodiments, the reactive multi-arm polymer further comprises at least one iodinated aromatic group.

In some embodiments, which can be used in conjunction with the above aspects and embodiments, the core region comprises at least one iodinated aromatic group.

In some embodiments, which can be used in conjunction with the above aspects and embodiments, the reactive multi-arm polymer further comprises at least one polymer arm that comprises at least one iodinated aromatic end group.

In some embodiments, which can be used in conjunction with the above aspects and embodiments, the reactive multi-arm polymer is formed by a process that comprises (a) reacting a multi-arm polymer that comprises three or more precursor polymer arms linked to the core region, at least three of the precursor polymer arms comprising the polyoxazoline segment and a hydroxyethyl end group, with an oxidizing agent that converts the hydroxyethyl end group to a carboxymethyl end group, and (b) reacting the reaction product of step (a) with an N-hydroxy cyclic imide compound in an ester coupling reaction to form the cyclic-imidyl-oxycarbonyl- end group.

In some embodiments, which can be used in conjunction with the above aspects and embodiments, the core region comprises a polyol residue.

In some embodiments, which can be used in conjunction with the above aspects and embodiments, the core region comprises an iodinated polyol residue.

In some embodiments, which can be used in conjunction with the above aspects and embodiments, the polyamino compound comprises a plurality of basic amino acid residues.

In some embodiments, which can be used in conjunction with the above aspects and embodiments, the polyamino compound is an iodinated polyamino compound.

In some embodiments, which can be used in conjunction with the above aspects and embodiments, the system comprises a first composition that comprises the polyamino compound in a first container and a second composition that comprises the reactive multi-arm polymer in a second container.

In some embodiments, which can be used in conjunction with the above aspects and embodiments, the system further comprises a delivery device.

In other aspects, the present disclosure pertains to a hydrolysis-resistant crosslinked hydrogel produced by covalent crosslinking between the reactive multi-arm polymer of any of the above aspects and embodiments, and the polyamino compound of any of the above aspects and embodiments.

In some embodiments, the hydrolysis-resistant crosslinked hydrogel has a radiopacity that is greater than 100 Hounsfield units (HU).

In other aspects, a method of treatment is provided that comprises administering to a subject a mixture that comprises the reactive multi-arm polymer of any of the above aspects and embodiments and the polyamino compound of any of the above aspects and embodiments, wherein the mixture is administered under conditions such that the polyamino compound and the reactive multi-arm polymer react to form a crosslinked hydrogel after administration.

In some embodiments, wherein the method of treatment comprises administering to the subject (a) a first fluid composition that comprises the polyamino compound and the reactive multi-arm polymer and (b) a second fluid composition that comprises an accelerant that accelerates reaction of the polyamino compound and the reactive multi-arm polymer.

The above and other aspects, embodiments, features and benefits of the present disclosure will be readily apparent from the following detailed description.

### BRIEF DESCRIPTION OF THE DRAWINGS

FIG. 1 schematically illustrates the formation of a hydroxyl-terminated 8-arm-polyoxazoline having a tripentaerythritol residue core, in accordance with an embodiment of the present disclosure.

FIG. 2 schematically illustrates the formation of a hydroxyl-terminated 8-arm-polyethylene oxide having a tripentaerythritol residue core, in accordance with an embodiment of the present disclosure.

FIG. 3 schematically illustrates the formation of a hydroxyl-terminated 3-arm-polyethylene oxide having a 1,3,5-triiodo-2,4,6-tris-hydroxymethylbenzene residue core, in accordance with an embodiment of the present disclosure.

FIG. 4 schematically illustrates the formation of a N-succinimidyloxycarbonyl-terminated multi-arm polyoxazoline, in accordance with an embodiment of the present disclosure.

FIG. 5 schematically illustrates the formation of an amino-terminated multi-arm-polyethylene oxide, in accordance with an embodiment of the present disclosure.

FIG. 6 schematically illustrates the formation of a N-succinimidyloxycarbonyl-terminated multi-arm polyethylene oxide, in accordance with another embodiment of the present disclosure.

FIG. 7 schematically illustrates the formation of a multi-arm-polyethylene oxide in which a first portion of the arms comprise iodinated end groups and a second portion of the arms comprise reactive N-succinimidyloxycarbonyl end groups, in accordance with an embodiment of the present disclosure.

FIG. 8 schematically illustrates the formation of a crosslinked polymer network by reaction of trilysine with the multi-arm polymer of FIG. 6, in accordance with an embodiment of the present disclosure.

FIG. 9 schematically illustrates the formation of a crosslinked polymer network by reaction of trilysine with the multi-arm polymer of FIG. 7, in accordance with an embodiment of the present disclosure.

FIG. 10 schematically illustrates a delivery device, in accordance with an embodiment of the present disclosure.

FIGS. 11A and 11B schematically illustrate the injection of a crosslinked polymer network into a bladder neck of a patient, in accordance with an embodiment of the present disclosure.

FIG. 12 illustrates a delivery device, in accordance with another embodiment of the present disclosure.

### DETAILED DESCRIPTION

In some aspects of the present disclosure, hydrolysis-resistant hydrogels are provided that comprises a crosslinked reaction product of (a) a polyamino compound and (b) a reactive polymer comprising reactive moieties that are reactive with the amino groups of the polyamino compound, wherein the crosslinked reaction product does not contain esters or other readily hydrolysable groups that would lead to breakdown of the crosslinked reaction product upon implantation. Such hydrogels have long-term stability once implanted, opening up a variety of medical applications.

As used herein, a “hydrogel” is a crosslinked polymer that contains water or can absorb water but does not dissolve when placed in water. As used herein, an implanted hydrogel has “long-term stability” if it undergoes less than 25 wt % bioresorption, preferably less than 10 wt %, over the course of at least 5 years, preferably 10 years, after implantation in subject.

Reactive polymers for use in the present disclosure include reactive multi-arm polymers that comprise a plurality of polymer arms linked to a core region, wherein each of the polymer arms comprise a hydrophilic polymer segment. In various embodiments, a first end of the hydrophilic polymer segment is covalently linked to the core region and a reactive moiety is covalently linked to a second end (opposite end) of the hydrophilic polymer segment.

Reactive polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms.

Hydrophilic polymer segments for the polymer arms can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Polymer segments for use in the multi-arm polymers of the present disclosure typically contain between 10 and 1000 monomer units or more.

Reactive moieties include moieties that comprise electrophilic groups.

Electrophilic groups may be selected, for example, from cyclic imide ester

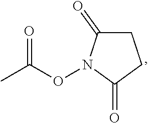

groups, such as succinimide ester groups, maleimide ester groups, glutarimide ester groups, diglycolimide ester groups, phthalimide ester groups, and bicyclo[2.2.1]hept-5-ene-2,3-dicarboxylic acid imide ester groups,

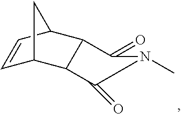

imidazole ester groups, imidazole carboxylate groups and benzotriazole ester groups, among other possibilities.

The electrophilic groups may be linked to the hydrophilic polymer segment through any suitable hydrolysis-resistant linking moiety, which may be selected, for example, from a linking moiety that comprises a C₁-C₆ alkyl group, a linking moiety that comprises an ether group, a linking moiety that comprises an amide group, a linking moiety that comprises a urethane group, a linking moiety that comprises a urea group, or a linking moiety that comprises a combination of two or more of the foregoing groups, among others.

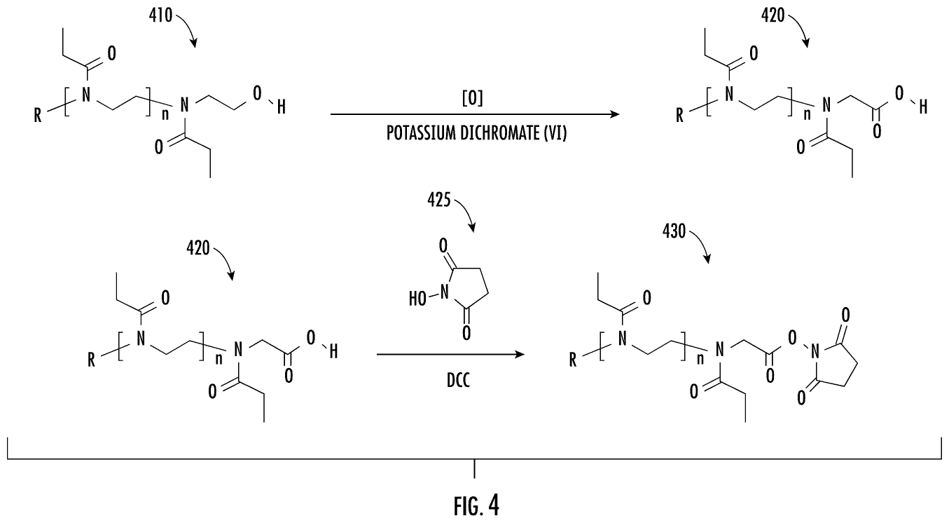

In some embodiments, the reactive moieties comprise cyclic-imidyl-oxycarbonyl-C₂-C₈-alkylene groups, including cyclic-imidyl-oxycarbonyl-dimethylene groups, cyclic-imidyl-oxycarbonyl-trimethylene groups, cyclic-imidyl-oxycarbonyl-tetramethylene groups, cyclic-imidyl-oxycarbonyl-pentamethylene groups, cyclic-imidyl-oxycarbonyl-hexamethylene groups, cyclic-imidyl-oxycarbonyl-heptamethylene groups, and cyclic-imidyl-oxycarbonyl-octamethylene groups. Specific examples include succinimidyl-oxycarbonyl-C₂-C₈-alkylene groups, maleimidyl-oxycarbonyl-C₂-C₈-alkylene groups, glutarimidyl-oxycarbonyl-C₂-C₈-alkylene groups, diglycolimidyl-oxycarbonyl-C₂-C₈-alkylene groups, phthalimidyl-oxycarbonyl-C₂-C₈-alkylene groups, and bicyclo[2.2.1]hept-5-ene-2,3-dicarboxylic-acid-imidyl-oxycarbonyl-C₂-C₈-alkylene groups, among other possibilities. In one specific embodiment described in conjunction with FIG. 4 below, the reactive moiety comprises a succinimidyloxycarbonylmethylene- group (or, alternatively, a succinimidyl acetate group).

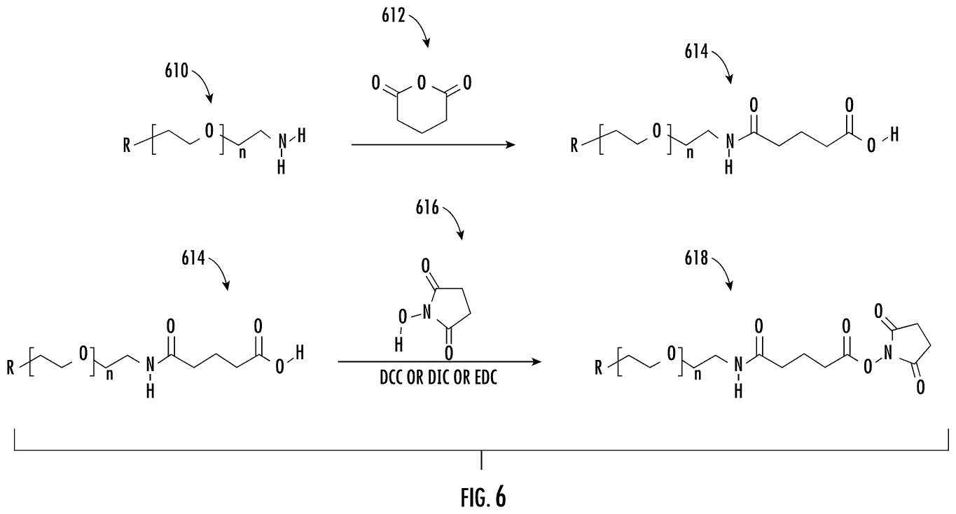

In some embodiments, the reactive moieties comprise cyclic-imidyl-oxycarbonyl-C₂-C₈-alkylenecarbonylamino groups, including succinimidyl-oxycarbonyl-C₂-C₈-alkylenecarbonylamino groups, maleimidyl-oxycarbonyl-C₂-C₈-alkylenecarbonylamino groups, glutarimidyl-oxycarbonyl-C₂-C₈-alkylenecarbonylamino groups, diglycolimidyl-oxycarbonyl-C₂-C₈-alkylenecarbonylamino groups, phthalimidyl-oxycarbonyl-C₂-C₈-alkylenecarbonylamino groups, and bicyclo[2.2.1]hept-5-ene-2,3-dicarboxylic-acid-imidyl-oxycarbonyl-C₂-C₈-alkylenecarbonylamino groups, among other possibilities. In one specific embodiment described in conjunction with FIG. 6 below, the reactive moiety comprises a succinimidyloxycarbonyltrimethylencarbonylamino- group (or, alternatively, a succinimidyl glutaramide group).

In the above and other embodiments, the cyclic imide ester group is linked to the hydrophilic polymer segment through a linkage that includes an alkylene chain that is two, three, four, five, six, or more carbon atoms in length, preferably 3 carbons in length. The inventors have found that varying the length of the alkylene chain in these embodiments is useful, for example, for tuning the rate at which the cyclic imide ester group reacts with an amino group of a polyamino compound to form a crosslinked reaction product as described below. In some embodiments, the inventors have found that increasing the number of carbon atoms in the alkyl chain results in a reduction in the reaction rate between the cyclic imide ester group and the amino group.

In certain embodiments, the core region comprises a residue of a non-iodinated or iodinated polyol comprising three or more hydroxyl groups, which is used to form the polymer arms. In certain beneficial embodiments, the core region comprises a residue of a polyol that contains from 3 to 100 hydroxyl groups, for example ranging anywhere from 3 to 4 to 5 to 6 to 7 to 8 to 9 to 10 to 11 to 12 to 15 to 20 to 25 to 50 to 75 to 100 hydroxy groups.

In some embodiments of the present disclosure, a non-iodinated or iodinated polyol such as one of those described below, among others, may be used to form a multi-functional initiator for polymer chain growth.

For example, the non-iodinated or iodinated polyol may be reacted with methanesulfonyl chloride to convert the hydroxyl groups into methanesulfonate groups, which can serve as leaving groups for the cationic polymerization of an oxazoline monomer to form polyoxazoline segments at the sites of each of the hydroxyl groups of the polyol. The resulting hydroxyl-terminated polyoxazoline segments possess tunable hydrophilicity depending on the desired water-solubility of the resulting multi-arm polymer, for example, with increasing polyoxazoline segment length leading to increased hydrophilicity in some embodiments.

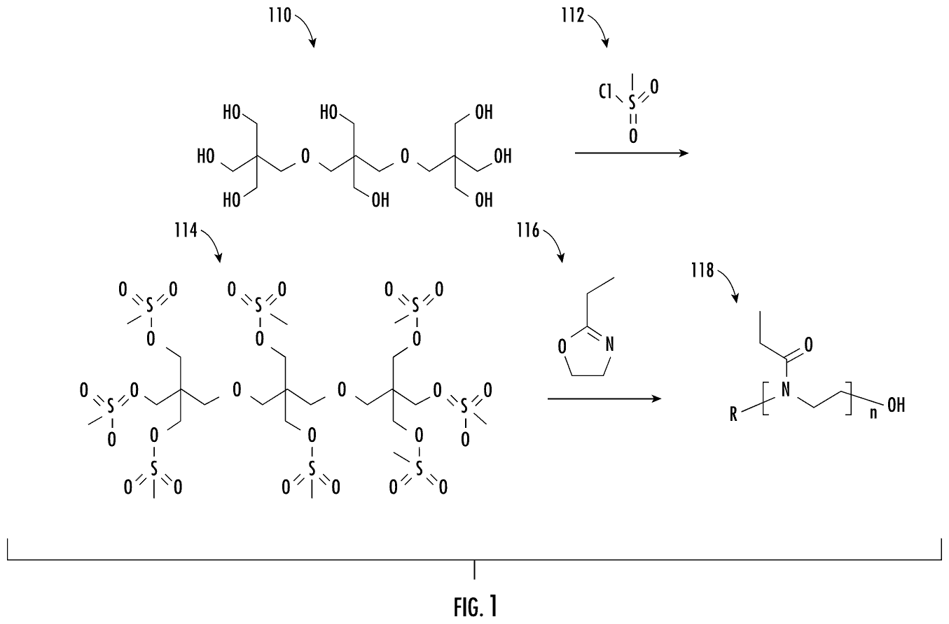

In a particular embodiment shown in FIG. 1, commercially available tripentaerythritol (110) is reacted with methanesulfonyl chloride (112) to convert the hydroxyl groups of the tripentaerythritol into methanesulfonate groups, thereby forming an octa-functional initiator (114), which can be used for the polymerization of 2-ethyl-2-oxazoline (116). The polymerization process leads to poly(2-ethyl-2-oxazoline) chain growth at sites previously occupied by each of the eight hydroxyl groups of the tripentaerythritol, forming a hydroxyl-terminated 8-arm-poly(2-ethyl-2-oxazoline) (118) having a tripentaerythritol residue core and eight poly(2-ethyl-2-oxazoline) arms. Each of the polyoxazoline arms has a terminal hydroxyl group. In FIG. 1, n is an integer representing the number of monomer units in each polymer segment shown.

As another example, a non-iodinated or iodinated polyol may be used as an initiator for ring-opening polymerization of ethylene oxide to form polyethylene oxide (PEO) segments (also referred to a polyethylene glycol (PEG) segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity.

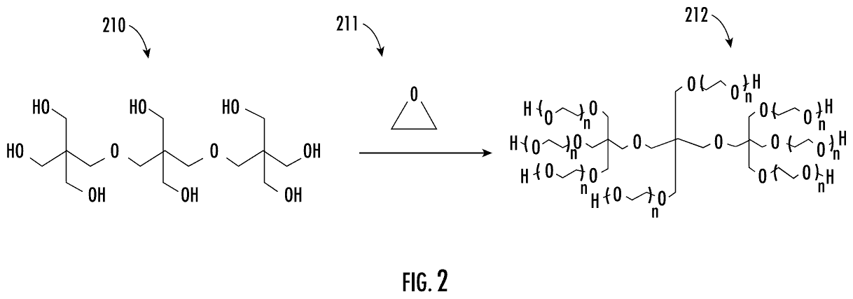

In a particular embodiment shown in FIG. 2, commercially available tripentaerythritol (210) is used as an octa-functional initiator, which undergoes ring-opening polymerization with ethylene oxide (211). The polymerization process leads to poly(ethylene oxide) chain growth at each of the eight hydroxyl groups of the tripentaerythritol, forming an 8-arm-PEG (212) having a tripentaerythritol residue core. Each of the PEG arms has a terminal hydroxyl group. In FIG. 2, n is an integer representing the number of monomer units in each polymer segment shown.

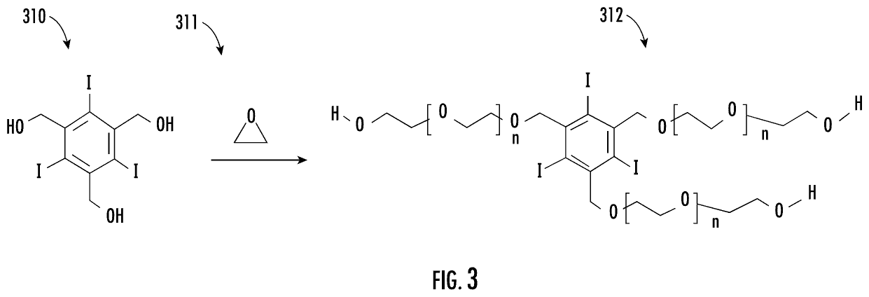

The strategies shown in FIGS. 1 and 2 can be used in conjunction with a wide range of polyols, including those described below. In another particular embodiment shown in FIG. 3, an iodinated polyol, specifically, 1,3,5-triiodo-2,4,6-tris-hydroxymethylbenzene (310), is used as an initiator, which undergoes ring-opening polymerization with ethylene oxide (311). The polymerization process leads to poly(ethylene oxide) (PEG) chain growth at each of the three hydroxyl groups of the 1,3,5-triiodo-2,4,6-tris-hydroxymethylbenzene (310). The resulting multi-arm polymer (312) contains three PEG arms that extend from a 1,3,5-triiodo-2,4,6-tris-hydroxymethylbenzene residue core. Each of the PEG arms has a terminal hydroxyl group. In FIG. 3, n is an integer representing the number of monomer units in each polymer segment shown.

Illustrative non-iodinated polyols may be selected, for example, from sugars (monosaccharides, disaccharides, trisaccharides, etc.), sugar alcohols, calixarenes, cyclodextrins, polyhydroxylated polymers, catechins, flavanols, anthocyanins, stilbenes, and polyphenols, among others.

Illustrative non-iodinated polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols. Specific examples include methane triol, glycerol, trimethylolpropane, benzenetriol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others.

Illustrative non-iodinated polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

Iodinated polyols are desirable where radiopacity is desired. Illustrative iodinated polyols include iodinated aromatic polyols, examples of which are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, ether groups, alkyl groups, and combinations thereof, among others.

Further illustrative iodinated polyols for use in the present disclosure, in addition to the 1,3,5-triiodo-2,4,6-tris-hydroxymethylbenzene described above, include iodinated polyols that are known for use as iodinated contrast agents, whose biocompatibility has been demonstrated to be reasonably well tolerated. Specific examples of iodinated polyols include commercially available 1,3,5-triiodo-2,4,6-trishydroxymethylbenzene (CAS #178814-33-0), iodixanol (CAS #92339-11-2), iotrolan (CAS #79770-24-4), iohexol (CAS #66108-95-0), ioversol (CAS #87771-40-2), iopamidol (CAS #60166-93-0), iohexol impurity J (CAS #76801-93-9), and iopromide (CAS #73334-07-3), among others.

Reactive multi-arm polymers in accordance with the present disclosure can be formed from hydroxyl-terminated multi-arm polymers through various synthetic routes.

For example, in some embodiments, in an initial step, hydroxyalkyl end groups of a hydroxyl-terminated multi-arm polymer can be oxidized to form a carboxylic-acid-terminated multi-arm polymer having arms that each comprise one or more carboxylic acid end groups. A reactive group may then be linked to the carboxylic acid groups of the carboxylic-acid-terminated multi-arm polymer.

In some these embodiments, an electrophilic group may be linked to the carboxylic-acid-terminated polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic acid groups of the carboxylic-acid-terminated polymer in the presence of a suitable coupling agent (e.g., a carbodiimide coupling agent, such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to a hydrophilic polymer segment. In this way, a variety of reactive ester groups can be formed.

In some embodiments, a carboxylic-acid-terminated polymer may be formed by oxidizing hydroxyalkyl end groups (e.g., C₂-C₆-hydoxyalkyl end groups) of a hydroxyl-terminated multi-arm polymer with an oxidizing agent such as potassium dichromate (VI) or potassium permanganate, thereby forming carboxyalkyl end groups (e.g., C₂-C₆-carboxyalkyl end groups) at the sites previously occupied by the hydroxyalkyl end groups.

In a particular embodiment shown in FIG. 4, a multi-arm polymer, which comprises a core region and a plurality of polyoxazoline arms (specifically, poly(2-ethyl-2-oxazoline) arms) having carboxylic acid end groups, is formed by oxidizing hydroxyalkyl end groups of a hydroxyl-terminated multi-arm polymer having a core region that comprises a polyol residue and multiple hydroxyl-terminated polyoxazoline arms. In particular, a hydroxyl-terminated 8-arm poly(2-ethyl-2-oxazoline) (410), where R comprises, for example, a tripentaerythritol or hexaglycerol residue and n ranges from 10 to 60 (note that only one polymer arm is shown, and R represents the polyol residue and the remaining arms), is oxidized by reaction with potassium dichromate (VI) to create a carboxylic-acid-terminated 8-arm poly(2-ethyl-2-oxazoline) (420). Then, in a subsequent step, the carboxylic-acid-terminated 8-arm polyoxazoline (420) is reacted with N-hydroxysuccinimide (425) in the presence of a coupling agent, specifically, N,N′-dicyclohexylcarbodiimide (DCC), to form a succinimide-ester-terminated 8-arm polyoxazoline, specifically, a succinimidyl-oxycarbonyl-N-propanoyl-methylamino-terminated 8-arm poly(2-ethyl-2-oxazoline) (430).

More generally, a process of this type can be used to form various cyclic-imidyl-oxycarbonyl-N—C₂-C₇-alkanoyl-methylamino- end groups for various multi-arm polymers having poly(2-C₁-C₆-alkyl-2-oxazoline) arms, for example, cyclic-imidyl-oxycarbonyl-N-ethanoyl-methylamino- end groups in the case of multi-arm polymer with poly(2-methyl-2-oxazoline) arms, cyclic-imidyl-oxycarbonyl-N-propanoyl-methylamino- end groups in the case of poly(2-ethyl-2-oxazoline) arms, cyclic-imidyl-oxycarbonyl-N-n-butanoyl-methylamino- end groups in the case of poly(2-n-propyl-2-oxazoline) arms, cyclic-imidyl-oxycarbonyl-N-isobutanoyl-methylamino- end groups in the case of poly(2-isopropyl-2-oxazoline) arms, cyclic-imidyl-oxycarbonyl-N-n-pentanoyl-methylamino- end groups in the case of poly(2-n-butyl-2-oxazoline) arms, and so forth.

Analogous processes can be performed with various other hydroxyl-terminated multi-arm polymers including those of FIG. 2 and FIG. 3.

As another example, in some embodiments, in an initial step, hydroxyl end groups of a hydroxyl-terminated multi-arm polymer converted into amino groups of an amino-terminated multi-arm polymer having arms that each comprise one or more amino end groups, which amino groups are then used to form reactive end groups.

For example, a multi-arm polymer that comprises a core region and a plurality of polymer arms with hydroxyalkyl end groups may be reacted with methanesulfonyl chloride to form a multi-arm polymer that comprises a core region and a plurality of polymer arms with methanesulfonate end groups. The methanesulfonate end groups may then be reacted with ammonia to form a multi-arm polymer that comprises a core region and a plurality of polymer arms with amino end groups.

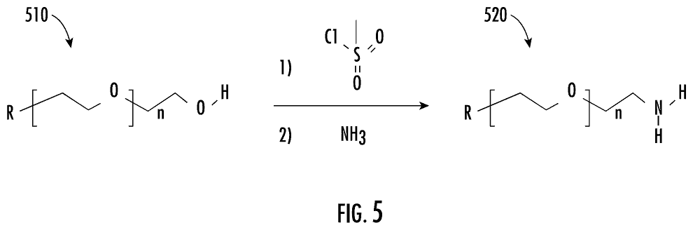

In a particular example shown in FIG. 5, a hydroxyl-terminated 8-arm PEG (510) where R comprises a tripentaerythritol or hexaglycerol residue and n ranges from 30 to 140 (only one polymer arm is shown; the polyol residue and the remaining seven arms are represented by R), is first treated with methanesulfonyl chloride to form a polymer in which hydroxyl groups of the 8-arm PEG (510) are converted into methanesulfonate groups. The methanesulfonate groups are then reacted with ammonia to convert the methanesulfonate groups to amino groups, thereby forming an amino-terminated 8-arm PEG (520).

Analogous processes can be performed with other hydroxyl-terminated multi-arm polymers, including those of FIG. 1 and FIG. 3.

After obtaining an amino-terminated multi-arm polymer, the amino-terminated multi-arm polymer may then be reacted with a cyclic anhydride in a ring opening reaction to form a carboxylic-acid-terminated multi-arm polymer in which the carboxylic acid groups are linked to the hydrophilic polymer segments of the polymer arms through an amide group.

Cyclic anhydrides for use in with the present disclosure include non-iodinated and iodinated cyclic anhydrides.

Non-iodinated cyclic anhydrides for use in the present disclosure include various cyclic anhydrides having a ring that comprises a —C₂-C₈-alkylene- group, including, for example, succinic anhydride,

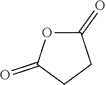

(CAS #108-30-5), which comprises a dimethylene group, glutaric anhydride,

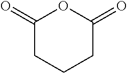

(CAS #108-55-4), which comprises a trimethylene group, adipic anhydride,

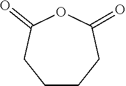

(CAS #2035-75-8), which comprises a tetramethylene group, pimelic anhydride,

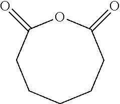

(CAS #10521-07-0), which comprises a pentamethylene group, and suberic anhydride (CAS #10521-06-9),

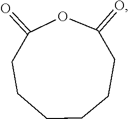 [SMILES: O=C1CCCCCCC(=O)O1]

which comprises a hexamethylene group, among others.

Iodinated cyclic anhydrides for use in the present disclosure include those that comprise a cyclic anhydride group, particularly, a cyclic anhydride group having a ring that comprises a —C₂-C₈-alkylene- group to which is linked one or more iodinated aromatic groups. Examples of iodinated aromatic groups include iodine-substituted monocyclic aromatic groups and iodine-substituted multicyclic aromatic groups which may contain, for example, six, ten, fourteen, eighteen or more carbon atoms, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms.

The one or more iodinated aromatic groups may, for example, may be linked to the cyclic anhydride group though a suitable linkage (e.g., a bond or a suitable linking moiety, which may contain, for example, an alkyl group, an ether group, an amide group, or a combination thereof, among others).

Examples of an iodinated cyclic anhydride in which an iodinated aromatic group is linked to cyclic anhydride group through a bond include 4-(2-iodophenyl)tetrahydropyran-2,6-dione,

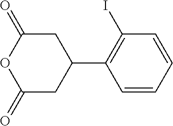 [SMILES: O=C1CC(c2ccccc2I)CC(=O)O1]

CAS #2354202-92-7, and 4-(2,3,5-triiodophenyl)tetrahydropyran-2,6-dione,

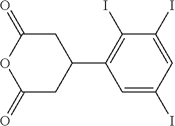 [SMILES: O=C1CC(c2cc(I)cc(I)c2I)CC(=O)O1]

(CAS #2357909-35-2), among others. An example of an iodinated cyclic anhydride in which an iodinated aromatic group is linked to cyclic anhydride group through a linking group is 4-((4-iodophenyl)methyl), tetrahydropyran-2,6-dione,

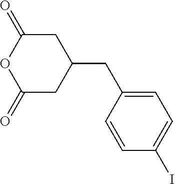 [SMILES: O=C1CC(Cc2ccc(I)cc2)CC(=O)O1]

(CAS #2354625-91-3), among others.

After forming a carboxylic-acid-terminated multi-arm polymer in which the carboxylic acid groups are linked to the hydrophilic polymer segments of the polymer arms through an amide group as described above, a reactive moiety comprising an electrophilic group may then be linked to the carboxylic-acid-terminated multi-arm polymer.

In particular, an electrophilic group may be formed at the site of each of the carboxylic acid groups of the carboxylic-acid-terminated multi-arm polymer.

For example, in particular embodiments where a cyclic imide ester group is employed as an electrophilic group, an N-hydroxy cyclic imide compound may be reacted with the carboxylic acid groups of the carboxylic-acid-terminated multi-arm polymer in an amide coupling reaction to form a reactive multi-arm polymer in which reactive cyclic imide ester groups are linked to the hydrophilic polymer segments of the polymer arms through a linkage that comprises an alkylene group, for example, a C₂-C₈-alkylene group (which is part of a residue of the cyclic anhydride) and an amide. For instance, an N-hydroxy cyclic imide compound (e.g., N-hydroxysuccinimide, N-hydroxymalcimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic acid groups of the carboxylic-acid-terminated polymer in the presence of a suitable coupling agent as described above. In this way, a variety of reactive multi-arm polymers can be formed.

In a particular example shown in FIG. 6, an amino-terminated 8-arm PEG (610) is reacted with a glutaric anhydride (612) in a ring opening reaction to form carboxylic-acid-terminated 8-arm PEG (614) in which the carboxylic acid end groups are linked the PEG segments through a three-carbon alkyl chain (i.e., a trimethylene group) and an amide group. Then, N-hydroxy succinimide (616) is reacted with the carboxylic acid groups of the carboxylic-acid-terminated 8-arm PEG (614) in an amide coupling reaction in the presence of a suitable coupling agent such as DCC, DIC or EDC, to form succinimide-ester-terminated 8-arm PEG (618) in which the succinimide ester groups are linked to the PEG segments of the multi-arm polymer through linkages that comprise a three-carbon alkyl chain (i.e., a trimethylene group) and an amide group.

As can be seen from FIG. 6, glutaric anhydride can be used to form cyclic-imidyl-oxycarbonyl-1,3-n-propylene-carbonylamino end groups, more specifically succinimidyl-oxycarbonyl-1,3-n-propylene-carbonylamino end groups. More generally, various cyclic anhydrides having a ring that comprises a —C₂-C₈-alkylene-group may be used to form various cyclic-imidyl-oxycarbonyl-C₂-C₆-alkylene-carbonylamino end groups. For example, (a) succinic anhydride can be used to form cyclic-imidyl-oxycarbonyl-1,2-ethylene-carbonylamino end groups, more specifically succinimidyl-oxycarbonyl-1,2-ethyl-carbonylamino end groups, (b) adipic anhydride can be used to form cyclic-imidyl-oxycarbonyl-1,4-n-butylene-carbonylamino end groups, more specifically succinimidyl-oxycarbonyl-1,4-n-butylene-carbonylamino end groups, (c) pimelic anhydride can be used to form cyclic-imidyl-oxycarbonyl-1,5-n-pentylene-carbonylamino end groups, more specifically succinimidyl-oxycarbonyl-1,5-n-pentylene-carbonylamino end groups, and (d) suberic anhydride can be used to form cyclic-imidyl-oxycarbonyl-1,6-n-hexylene-carbonylamino end groups, more specifically succinimidyl-oxycarbonyl-1,6-n-hexylene-carbonylamino end groups, among other possibilities.

In other embodiments, a first portion of the polymer arms of an amino-terminated multi-arm polymer may be reacted in an amide coupling reaction with a carboxylic-acid-containing iodinated compound such that the first portion of the polymer arms are terminated with iodinated end groups. This first portion may range, for example, from 5 to 90%, typically from 10 to 45%, of the amino-terminated polymer arms in the reaction mixture. This reaction is typically conducted in the presence of a suitable coupling agent such as a carbodiimide coupling agent.

Moreover, a second portion of the polymer arms of the amino-terminated multi-arm polymer may be reacted with cyclic anhydride in a ring opening reaction such that the second portion of the polymer arms are terminated with carboxylic acid groups, followed by the formation of an electrophilic group at a site of each of the carboxylic acid groups, as described above. This second portion may range, for example, from 5 to 90%, typically from 55 to 90%, of the amino-terminated polymer arms in the reaction mixture.

Carboxylic-acid-containing iodinated compounds for use in the present disclosure include triiodobenzoic acid

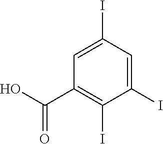 [SMILES: O=C(O)c1cc(I)cc(I)c1I]

(CAS #88-82-4), diatrizoic acid,

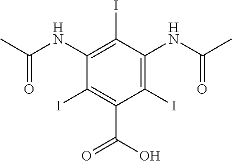 [SMILES: CC(=O)Nc1c(I)c(NC(C)=O)c(I)c(C(=O)O)c1I]

(CAS #117-96-4), N-acetyl-3,5-diiodo-L-tyrosine,

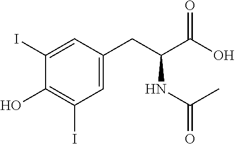 [SMILES: CC(=O)N[C@@H](Cc1cc(I)c(O)c(I)c1)C(=O)O]

(CAS #1027-28-7), N-acetyl-3-diiodo-L-tyrosine,

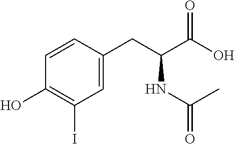 [SMILES: CC(=O)N[C@@H](Cc1ccc(O)c(I)c1)C(=O)O]

(CAS #1023-47-8) and N-acetyl-thyroxine,

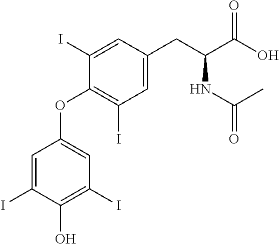 [SMILES: CC(=O)N[C@@H](Cc1cc(I)c(Oc2cc(I)c(O)c(I)c2)c(I)c1)C(=O)O]

(CAS #26041-51-0), among others.

Various carboxylic-acid-containing iodinated compounds, along with their CAS numbers, are listed in the following table.

| | | # I |
|---|---|---|
| Name | CAS | atoms |
| 2-Iodobenzoic acid | 88-67-5 | 1 |
| 4-Iodobenzoic acid | 619-58-9 | 1 |
| 3-Iodobenzoic acid | 618-51-9 | 1 |
| 4-Iodopicolinic acid | 405939-79-9 | 1 |
| 3,5-Diiodobenzoic acid | 19094-48-5 | 2 |
| 3,4-Diiodobenzoic acid | 35674-20-5 | 2 |
| 3,5-Diiodo-4-(4-methoxyphenoxy)benzoic acid | 34043-77-1 | 2 |
| Diatrizoate | 117-96-4 | 3 |
| 2,3,5-Triiodobenzoic acid | 88-82-4 | 3 |
| Acetrizoic acid | 85-36-9 | 3 |
| Metrizoate | 1949-45-7 | 3 |
| 3,4,5-Triiodobenzoic acid | 2338-20-7 | 3 |
| Ioxitalamic acid | 28179-44-4 | 3 |
| Iothalamic acid | 2276-90-6 | 3 |
| 2,4,6-Triiodo-1,3,5-benzenetricarboxylic acid | 79211-41-9 | 3 |
| 2,4,6-Triiodobenzoic acid | 2012-31-9 | 3 |
| enzoic acid, 3-[[[2-hydroxy-1- | 87932-11-4 | 3 |
| (hydroxymethyl)ethyl]amino]carbonyl]-5-[(2- | | |
| hydroxy-1-oxopropyl)amino]-2,4,6-triiodo-, (S)- | | |
| (9CI) | | |
| Ioseric acid | 51876-99-4 | 3 |
| 4-[4-(Acetyloxy)-3-iodophenoxy]-3,5- | 2260-084 | 3 |
| diiodobenzoic acid | | |
| Ioglicic acid | 49755-67-1 | 3 |
| 2,4,6-Triiodo-3-[(1-oxo-3,6,9,12,15- | 16024-67-2 | 3 |
| pentaoxahexadec-1-yl)amino]benzoic acid | | |
| 3-[[(1,1-Dimethylethoxy)carbonyl]amino]-2,4,6- | 2358047-48-8 | 3 |
| triiodobenzoic acid | | |
| 2,3,4,6-Tetraiodobenzoic acid | 71463-71-3 | 4 |
| Tetraform | 2055-97-2 | 4 |
| 2,3,5,6-Tetraiodo-1,4-benzenedicarboxylic acid | 7606-84-0 | 4 |
| N-Acetyl-O-(4-hydroxy-3,5-diiodophenyl)-3,5- | 26041-51-0 | 4 |
| diiodo-L-tyrosine | | |
| D-Tyrosine, N-[(1,1-dimethylethoxy)carbonyl]- | 89624-64-6 | 4 |
| O-(4-hydroxy-3,5-diiodophenyl)-3,5-diiodo- | | |
| (9CI, ACI) | | |
| 2,3,4,5,6-Pentaiodobenzoic acid | 64385-02-0 | 5 |
| Ioxaglic acid | 59017-64-0 | 6 |
| Ioglycamic acid | 2618-25-9 | 6 |
| Iocarmic acid | 10397-75-8 | 6 |

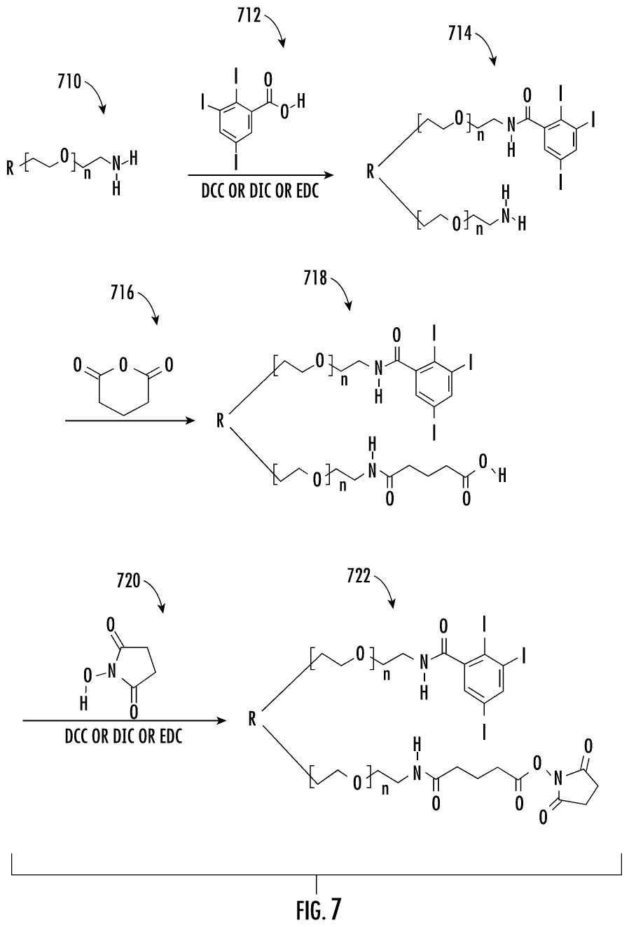

In a particular example shown in FIG. 7, a first portion of the polymer arms of an amino-terminated 8-arm PEG (710) are reacted with a carboxylic-acid-containing iodinated compound, specifically, 2,3,5-triiodobenzoic acid (712) in an amide coupling reaction in the presence of a suitable coupling agent such as DCC, DIC or EDC, to form 8-arm PEG (714) in which the first portion of the polymer arms are terminated with 2,3,5-triiodobenzamide groups and a second portion of the polymer arms remain terminated with amino groups. In a next step, the remaining amino groups of the second portion of the polymer arms are reacted with glutaric anhydride (716) in a ring opening reaction to form 8-arm PEG (718) in which the first portion of the polymer arms are terminated with 2,3,5-triiodobenzamide groups and the second portion of the polymer arms are terminated with carboxylic acid groups, which are linked the PEG segments through a trimethylene group and an amide group. Then, N-hydroxy succinimide (720) is reacted with the carboxylic acid groups of the 8-arm PEG (718) in an amide coupling reaction in the presence DCC, DIC or EDC, to form 8-arm PEG (722) in which the first portion of the polymer arms are terminated with 2,3,5-triiodobenzamide groups and the second portion of the polymer arms are terminated with succinimide ester groups that are linked to the PEG segments multi-arm polymer through linkages that comprise a trimethylene group and an amide group. Note that only two arms of the multi-arm polymer are schematically illustrated in FIG. 7, one representing the iodinated arms and the other representing the reactive arms.

As previously noted, in some aspects, the present disclosure provides hydrolysis-resistant crosslinked hydrogels that comprise a hydrolysis-resistant crosslinked reaction product of (a) a polyamino compound and (b) a reactive polymer comprising reactive moieties that are reactive with amino groups of the polyamino compound.

Such polyamino compounds include non-iodinated polyamino compounds and iodinated polyamino compounds.

Non-iodinated polyamino compounds suitable for use in the present disclosure include, for example, polyamines that contain at least two amino (—NH₂) groups (e.g., 2, 3, 4, 5, 6, 7, 8, 9, 10 or more amino groups in some embodiments). Non-iodinated polyamino compounds suitable for use in the present disclosure include those that comprise a plurality of —(CH₂)ₓ—NH₂ groups where x is 0, 1, 2, 3, 4, 5 or 6. Non-iodinated polyamino compounds suitable for use in the present disclosure include polyamino compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Further particular examples of non-iodinated polyamino compounds which may be used as the multifunctional compound include ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, and poly(allyl amine), among others.

Where additional radiopacity is desired in the crosslinked hydrogel, iodinated polyamine compounds may be employed.

In some embodiments, the iodinated polyamino compounds comprise a polyamino moiety that is linked to a carboxy-substituted iodinated moiety through an amide group. The iodinated polyamino compounds may comprise peptide oligomers that contain from 2 to 10 lysine and/or ornithine amino acid residues and one or more iodinated amino acid residues. The carboxy-substituted iodinated moieties may comprise iodinated amino acid residues.

Examples of iodinated amino acid residues include iodinated alpha-amino acid residues, iodinated beta-amino acid residues, iodinated gamma-amino acid residues and iodinated delta-amino acid residues. Examples of iodinated amino acid residues include amino acid residues that comprise an iodinated aromatic group. Examples of iodinated aromatic groups include iodo-phenyl groups and iodo-naphthyl groups. In particular embodiments, the iodinated amino acid residues include amino acid residues that comprise a hydroxy-iodo-aromatic group, such as a hydroxy-iodo-phenyl group or a hydroxy-iodo-naphthyl group. More particular examples of hydroxy-iodo-aromatic groups include hydroxy-iodo-phenyl groups selected from a mono-hydroxy-mono-iodo-phenyl group, a mono-hydroxy-di-iodo-phenyl group, a mono-hydroxy-tri-iodo-phenyl group, a mono-hydroxy-tetra-iodo-phenyl group, a di-hydroxy-mono-iodo-phenyl group, a di-hydroxy-di-iodo-phenyl group, a di-hydroxy-tri-iodo-phenyl group, a tri-hydroxy-mono-iodo-phenyl group, a tri-hydroxy-di-iodo-phenyl group, as previously indicated.

Specific examples of iodinated amino acid residues include residues of the following iodinated amino acids: iodo-phenylalanine, which

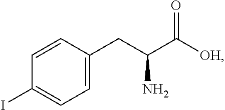 [SMILES: N[C@@H](Cc1ccc(I)cc1)C(=O)O]

which comprises a mono-iodo-phenyl group, monoiodotyrosine,

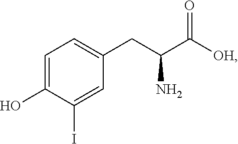 [SMILES: N[C@@H](Cc1ccc(O)c(I)c1)C(=O)O]

which comprises a mono-iodo-phenyl group, specifically, a mono-hydroxy-mono-iodo-phenyl group, diiodotyrosine,

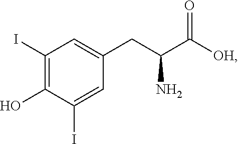 [SMILES: N[C@@H](Cc1cc(I)c(O)c(I)c1)C(=O)O]

which comprises a mono-hydroxy-di-iodo-phenyl group, diiodothyronine,

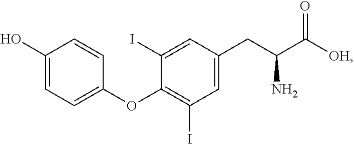 [SMILES: N[C@@H](Cc1cc(I)c(Oc2ccc(O)cc2)c(I)c1)C(=O)O]

which comprises a di-iodo-phenyl group and a hydroxy-phenyl group, triiodothyronine also known as T3,

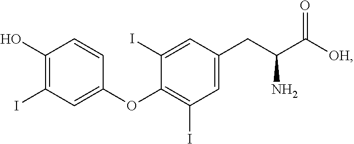 [SMILES: N[C@@H](Cc1cc(I)c(Oc2ccc(O)c(I)c2)c(I)c1)C(=O)O]

which comprises a di-iodo-phenyl group and a mono-hydroxy-mono-iodo-phenyl group, tetraiodothyronine also known as thyroxine or T4,

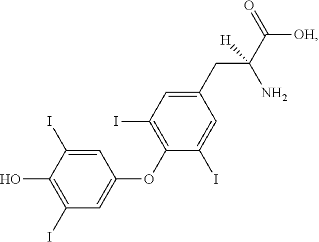 [SMILES: N[C@@H](Cc1cc(I)c(Oc2cc(I)c(O)c(I)c2)c(I)c1)C(=O)O]

which comprises a di-iodo-phenyl group and a mono-hydroxy-di-iodo-phenyl group, iodo-phenylalanine, and 6-iodo-L-DOPA, which comprises a di-hydroxy-mono-iodo-phenyl group, among others.

Iodinated polyamino compounds may be formed by an amide coupling reaction between (a) an iodinated amino acid derivative, for example, a C₁-C₅-alkyl ester of an iodinated amino acid, preferably a methyl ester of an iodinated amino acid, and (b) a carboxyl-substituted polyamino compound, selected for example, from these described below (after protection of the amino groups). Examples of such iodinated amino acid derivatives include C₁-C₅-alkyl esters of any of the preceding iodinated amino acids. After coupling, the protective groups on the residue of the carboxyl-substituted polyamino compound are removed and the C₁-C₅-alkyl ester may be converted into the corresponding carboxylic acid or anionic carboxylate group, thereby providing the final iodinated polyamino compound.

In various embodiments, the iodinated polyamino compounds comprise a polyamino moiety having a plurality (two, three, four, five, six, seven, eight, nine, ten or more) amino groups. For example, the polyamino moiety may comprises a plurality of (two, three, four, five, six, seven, eight, nine, ten or more) —(CH₂)ₓ—NH₂ groups where x is 0, 1, 2 3, 4, 5 or 6. In some of these embodiments, the polyamino moiety may comprises a plurality of —(CH₂)ₓ—NH₂ groups disposed along a polymeric moiety (defined here as a moiety comprising 2, 3, 4, 5, 6, 7, 8, 9, 10 or more monomer residues).

In some embodiments, the polyamino moiety of the iodinated polyamino compounds may correspond to a residue of a carboxyl-substituted polyamino compound (a compound comprising a carboxyl group and a plurality of amino groups). Examples of carboxyl-substituted polyamino compounds include peptides containing from 2 to 10 lysine and/or ornithine amino acid residues, including polylysines (e.g., dilysine, trilysine, tetralysine, pentalysine, etc.) and carboxyl-terminated polyamines such as carboxyl-terminated poly(allyl amine), carboxyl-terminated poly(vinyl amine), or carboxyl-terminated chitosan.

Commercially available examples of carboxyl-substituted polyamino compounds also include 16-amino-3-[2-[(4-aminobutyl) (3-aminopropyl)amino]-2-oxoethyl]-12-(3-aminopropyl)-6,9-bis(carboxymethyl)-11-oxo-3,6,9,12-tetraazahexadecanoic acid, L-ornithyl-L-ornithyl-L-ornithine, N²-[1-[N²—[N—(N-L-valyl-L-alanyl)-L-lysyl]-L-lysyl]-L-prolyl]-L-Lysine, L-Lysyl-L-tryptophyl-L-lysyl-L-lysine, N²,N⁵,N⁵-tris(3-aminopropyl)-L-ornithine, L-lysyl-L-ornithyl-L-lysine. D-lysyl-D-lysyl-D-lysine, glycylglycyl-L-lysylglycylglycyl-L-lysine, N²—[N⁴—[N—[N—(N-glycylglycyl)glycyl]glycyl]-L-lysyl]-L-lysine, L-Lysyl-L-threonyl-L-lysyl-L-lysine, glycylglycyl-L-lysyl-L-lysylglycyl-L-cysteine, L-lysyl-L-arginyl-L-lysyl-L-lysine, L-arginyl-L-lysyl-L-lysyl-L-lysine. L-leucyl-L-lysyl-L-seryl-L-lysyl-L-lysine, L-alanyl-L-methionylglycyl-L-lysyl-L-lysyl-L-lysine, L-lysyl-L-lysyl-L-lysyl-L-arginyl-L-glutamine, L-seryl-L-isoleucyl-L-lysyl-L-lysyl-Llysyl-L-lysine, N²—(N²-L-ornithyl-L-lysyl)-L-lysine, lysyllysyl-lysine, and L-lysyl-L-lysyl-L-lysyl-L-alanine.

Iodinated polyamino compounds may be formed by a reaction in which the carboxyl group of a carboxyl-substituted polyamino compound such as those described above (after protecting the amino groups of the carboxyl-substituted polyamino compound with a suitable protective agent, e.g., by reaction with di-tert-butyl decarbonate or another protective agent) is reacted with the amino group of an iodinated amino acid C₁-C₅-alkyl ester such as those described above, to form an amide bond between the two residues. A particular compound formed, for example, by amide coupling between tBoc-protected trilysine and diiodotyrosine methyl ester is shown here (after tBoc deprotection and hydrolysis of the methyl ester):

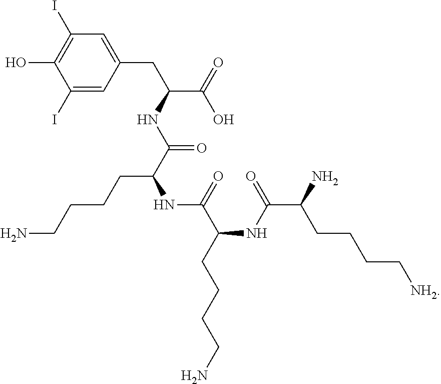 [SMILES: NCCCC[C@H](NC(=O)[C@H](CCCCN)NC(=O)[C@@H](N)CCCCN)C(=O)N[C@@H](Cc1cc(I)c(O)c(I)c1)C(=O)O]

Other examples of iodinated polyamino compounds include those formed from polyhydroxylated iodinated compounds by substituting hydroxyl groups of the polyhydroxylated iodinated compounds with amino groups (—NH₂ groups) by various suitable techniques. In one process, analogous to FIG. 5, hydroxyl groups of a polyhydroxylated iodinated compound are converted into methanesulfonate groups, followed by reaction with ammonia to convert the methanesulfonate groups to amino groups. As another example, hydroxyl groups of a polyhydroxylated iodinated compound are converted into methanesulfonate groups, followed by reaction with sodium amide, NaNH₂ (also known as sodium azanide) to convert the methanesulfonate groups to amino groups.

Using these and other techniques, a variety of iodinated polyamino compounds may be formed from iodinated polyhydroxylated compounds by amino substitution of all or a portion of the hydroxyl groups of the iodinated polyhydroxylated compounds. Such iodinated polyamino compounds may contain two, three, four, five, six, seven, eight, nine, ten, eleven, twelve or more amino groups.

Particular examples of iodinated polyamino compounds include the following, among others: iopromide, wherein at least a portion of the hydroxyl groups, specifically, four hydroxyl groups, have been substituted by amino groups, with the result being N,N′-Bis(2,3-diaminopropyl)-2,4,6-triiodo-5-(2-methoxyacetamido)-N-methylisophthalamide,

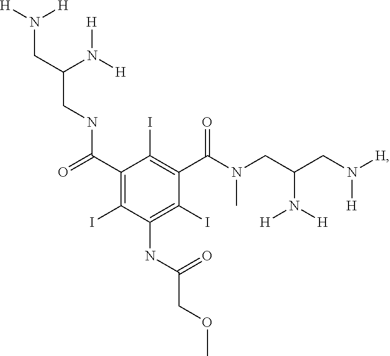

iopamidol, wherein at least a portion of the hydroxyl groups, specifically, five hydroxyl groups, have been substituted by amino groups, with the result being N,N′-bis(2-amino-1-(aminomethyl)ethyl)-2,4,6-triiodo-5-lactamidoisophthalamide

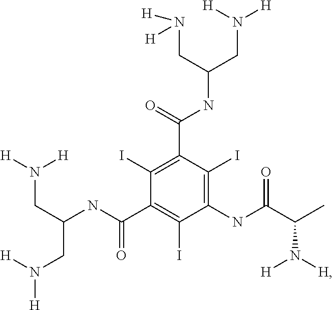

iomeprol, wherein at least a portion of the hydroxyl groups, specifically, five hydroxyl groups, have been substituted by amino groups, with the result being N,N′-bis(2,3-aminopropyl)-5-(2-amino-N-methylacetamido)-2,4,6-triiodoisophthalamide,

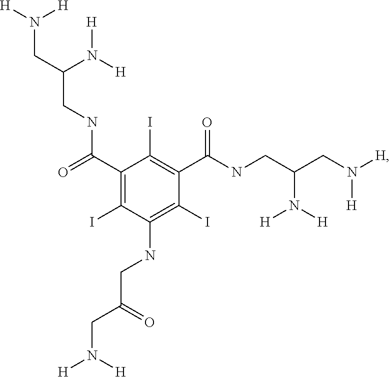

ioversol, wherein at least a portion of the hydroxyl groups, specifically, six hydroxyl groups, have been substituted by amino groups, with the result being N,N′-bis(2,3-diaminopropyl)-5-(2-amino-N-(2-amino) acetamido)-2,4,6-triiodoisophthalamide,

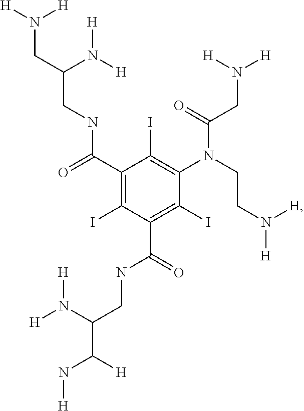

iobitridol, wherein at least a portion of the hydroxyl groups, specifically, six hydroxyl groups, have been substituted by amino groups, with the result being N,N′-bis(2,3-diaminopropyl)-5-((5-amino-2-(aminomethyl)-1-oxopropyl)amino)-2,4,6-triiodo-N,N′-dimethyl-1,3-benzenedicarboxamide,

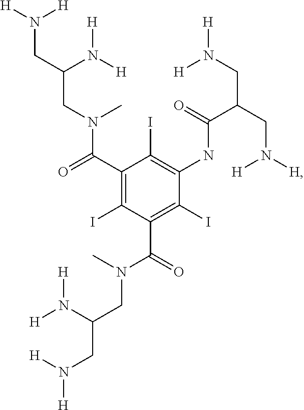

iodixanol, wherein at least a portion of the hydroxyl groups, specifically, eight out of nine hydroxyl groups, have been substituted by amino groups, with the result being 5,5′-((2-hydroxytrimethylene)bis(acetylimino))bis(N,N′-bis(2,3-diaminopropyl)-2,4,6-triiodoisophthalamide,

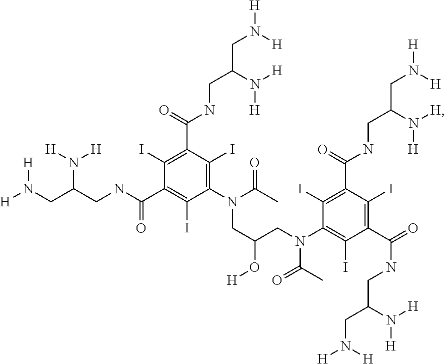

and iotrolan, wherein at least a portion of the hydroxyl groups, specifically, twelve hydroxyl groups, have been substituted by amino groups, with the result being 5,5′-((1,3-dioxo-1,3-propanediyl)bis(methylimino))bis(N,N′-bis(2,3-diamino-1-(aminomethyl) propyl)-2,4,6-triiodo-1,3-benzenedicarboxamide,

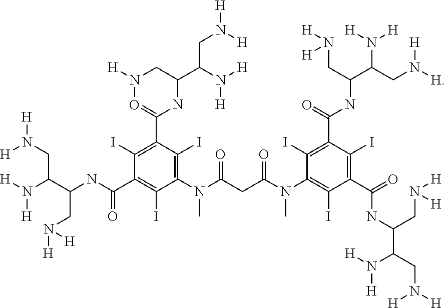

In some embodiments, the iodinated polyamino compound need not be formed from an iodinated polyhydroxylated compound. One example of such an iodinated polyamino compound is CA⁴⁺:

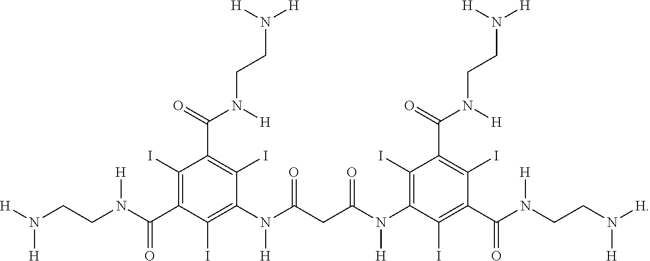

As previously noted, in some aspects, the present disclosure provides hydrogels that comprise a hydrolysis-resistant crosslinked reaction product of (a) a polyamino compound, such as those described above, and (b) a reactive polymer comprising reactive moieties that are reactive with amino groups of the polyamino compound, such as those described above.

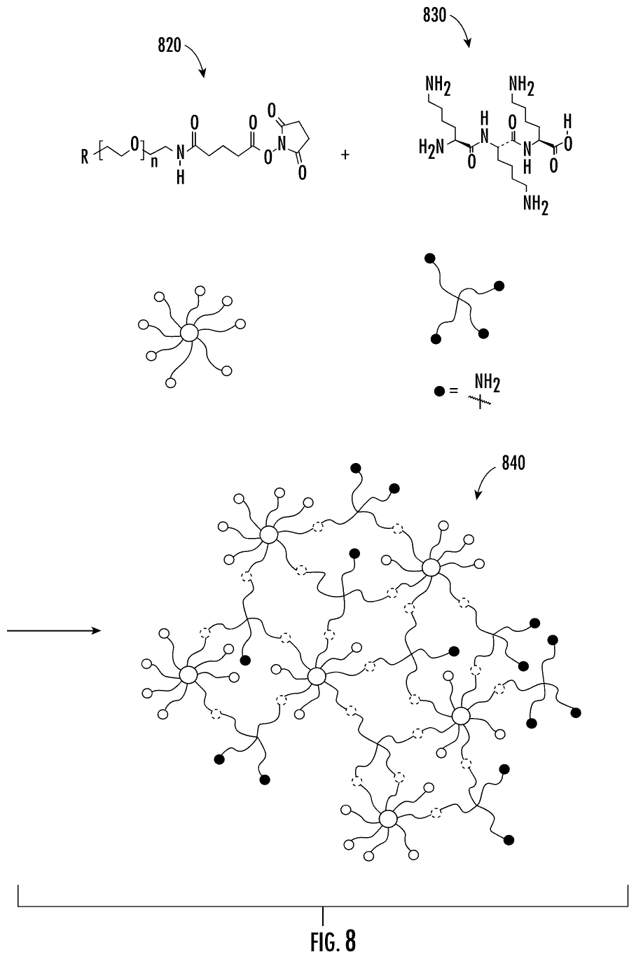

A specific embodiment is shown in FIG. 8, which schematically illustrates a crosslinking reaction between a reactive polymer (820) like that of FIG. 6 and a polyamino compound, specifically, trilysine (830). The reactive polymer (820) and the polyamino compound (830) are combined under conditions such that the succinimidyl ester groups of the reactive polymer (820) react with the amino groups of the polyamino compound (830) to form amide bonds accompanied by the release of N-hydroxysuccinimide, with the result being a crosslinked polymer network (840). The amide bond formation eliminates the ester group of the reactive polymer (820), with the result being an ester-free amide linkage such that the crosslinked polymer network (840) is resistant to hydrolysis. In certain embodiments, reaction between the succinimidyl ester groups and the amino groups is conducted at slightly basic pH (e.g., having a pH value ranging from 7.4 to 11) where the amino groups are deprotonated/neutrally charged and amide bond formation can occur spontaneously at body temperature.

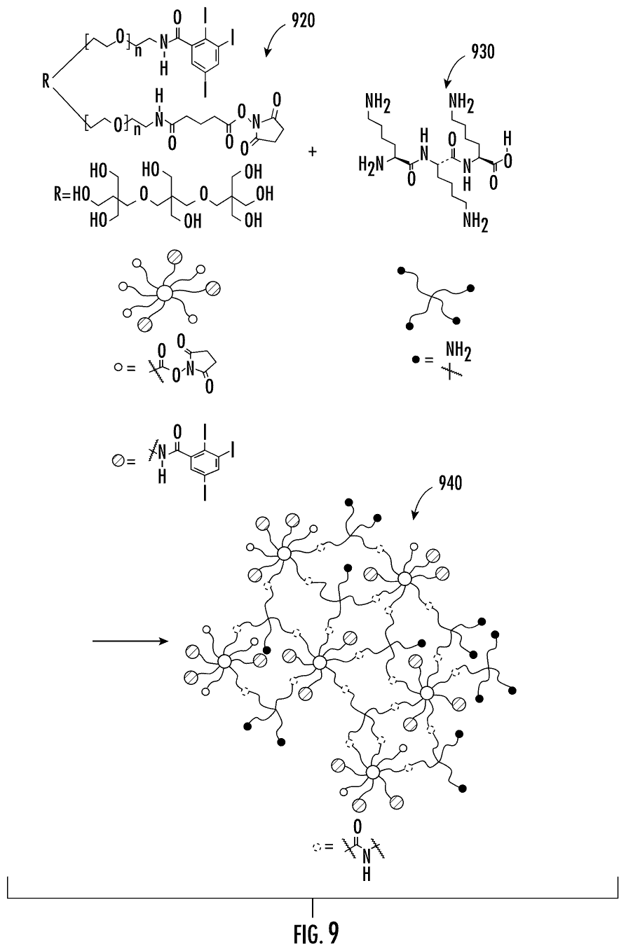

Another specific embodiment is shown in FIG. 9, which schematically illustrates a crosslinking reaction between the reactive polymer (920) like that of FIG. 7 and a polyamino compound, specifically, trilysine (930). As in FIG. 8, the reactive polymer (920) and the polyamino compound (930) are combined under conditions such that the succinimidyl ester groups of the reactive polymer (920) react with the amino groups of the polyamino compound (930) to form amide bonds accompanied by the release of N-hydroxysuccinimide, with the result being a crosslinked polymer network (940).

It will be appreciated that the crosslinking density of the hydrolysis-resistant crosslinked hydrogels described herein can be tuned, for example, (a) by varying the number of amino groups in the polyamino compound, (b) by varying the number of arms of the reactive multi-arm polymer, or (c) both.

In some embodiments, the hydrolysis-resistant crosslinked hydrogels of the present disclosure are visible under fluoroscopy. The hydrolysis-resistant crosslinked hydrogels may have a radiopacity that is greater than 100 Hounsfield units (HU), beneficially ranging anywhere from 100 HU to 250 HU to 500 HU to 750 HU to 1000 HU or more (in other words, ranging between any two of the preceding numerical values), for example, when measured on bench-top micro CT systems such as Xtreme CT from Scanco Medical (Wangen-Brüttisellen, Switzerland) or similar.

In some aspects of the present disclosure, a system is provided that comprises (a) a first composition that comprises a polyamino compound as described herein and (b) a second composition that comprises a reactive polymer comprising reactive moieties as described herein, wherein the system is configured to deliver the reactive polymer and the polyamino compound under conditions such that covalent crosslinks are formed between the reactive polymer and the polyamino compound.

The first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise additional agents, including therapeutic agents, imaging agents, colorants, tonicity adjusting agents, suspension agents, wetting agents, and pH adjusting agents as described below. The second composition may be a second fluid composition comprising the reactive polymer comprising reactive electrophilic moieties or a second dry composition that comprises the reactive polymer comprising reactive electrophilic moieties, to which a suitable fluid such as water for injection, saline, etc. can be added to form a second fluid composition. In addition to the reactive polymer comprising reactive electrophilic moieties, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the polyamino compound with a second fluid comprising the reactive polymer. Upon mixing the first and second fluid compositions, the polyamino compound crosslinks with the reactive polymer, forming a crosslinked product. The first and second fluid compositions may be combined to form hydrolysis-resistant crosslinked hydrogels.

In some embodiments, the polyamino compound is initially combined with the reactive polymer under conditions where crosslinking between the reactive polymer and the polyamino compound is suppressed (e.g., an acidic pH in the case where the reactive polymer comprises reactive electrophilic moieties). Then, when crosslinking is desired, the conditions are changed such that crosslinking is increased (e.g., a change from an acidic pH to a basic pH, in some embodiments), leading to crosslinking between the polyamino compound and the reactive polymer, thereby forming a crosslinked product. The first and second fluid compositions may be combined to form hydrolysis-resistant crosslinked hydrogels.

In some of these embodiments, the system comprises (a) a first composition that comprises a polyamino compound as described hereinabove, (b) a second composition that comprises a reactive polymer comprising reactive electrophilic moieties as described hereinabove, and (c) a third composition, specifically, an accelerant composition, that contains an accelerant that is configured to accelerate a crosslinking reaction between the polyamino compound and the reactive polymer comprising reactive electrophilic moieties.

The first composition may be a first fluid composition comprising the polyamino compound that is buffered to an acidic pH or a first dry composition that comprises the polyamino compound, to which a suitable fluid such as water for injection, saline, an acidic buffer solution, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 6.5, typically, from about 3 to about 5. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer comprising reactive electrophilic moieties or a second dry composition that comprises the reactive polymer comprising reactive electrophilic moieties from which a fluid composition is formed, for example, by the addition of a suitable fluid such as water for injection, saline, or the first fluid composition comprising the polyamino compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyamino compound that is buffered to an acidic pH and the second composition comprises a dry composition that comprises the reactive polymer comprising reactive electrophilic moieties. The first composition may then be mixed with the second composition to provide a prepared fluid composition that is buffered to an acidic pH and comprises the polyamino compound and the reactive polymer. In a particular example, a syringe may be provided that contains the first fluid composition comprising the polyamino compound that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that is buffered to an acidic pH and contains the polyamino compound and the reactive polymer, which can be withdrawn back into the syringe for administration.

The accelerant composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8.5 to about 12, typically, from about 9 to about 11. In addition to the above, the fluid accelerant composition may further comprise additional agents, including those described below.

A prepared fluid composition that is buffered to an acidic pH and comprises the polyamino compound and the reactive polymer comprising reactive electrophilic moieties as described above, and a fluid accelerant composition that is buffered to basic pH as described above, may be combined to form hydrolysis-resistant crosslinked hydrogels.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises a polyamino compound as described herein and a second reservoir that contains a second fluid composition that comprises a reactive polymer as described herein, wherein the first and second fluid compositions form a crosslinked product upon mixing.

In some embodiments, the system may include a delivery device that comprises a first reservoir that contains a first fluid composition that comprises a polyamino compound as described herein and a reactive polymer comprising reactive electrophilic moieties as described herein and is buffered to an acidic pH, such as the prepared fluid composition previously described, and a second reservoir that contains second fluid composition, such as the fluid accelerant composition described herein.

In either case, during operation, the first fluid composition and the second fluid composition are dispensed from the first and second reservoirs and combined, whereupon the polyamino compound and the reactive polymer and crosslink with one another to form a hydrolysis-resistant crosslinked hydrogel.

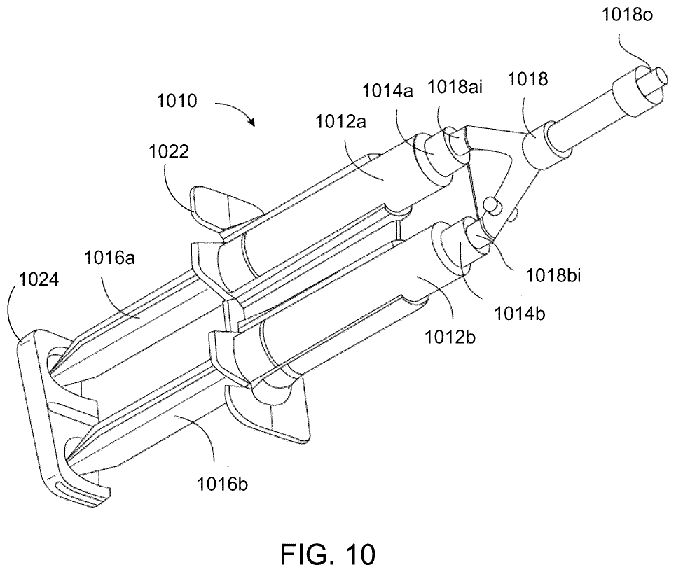

In particular embodiments, and with reference to FIG. 10, the system may include a delivery device 1010 that comprises a double-barrel syringe, which includes a first barrel 1012a having a first barrel outlet 1014a, which first barrel contains a first fluid composition as described above, a first plunger 1019a that is movable in the first barrel 1012a, a second barrel 1012b having a second barrel outlet 1014b, which second barrel 1012b contains a second fluid composition as described above, and a second plunger 1019b that is movable in the second barrel 1012b. In some embodiments, the device 1010 may further comprise a mixing section 1018 having a first mixing section inlet 1018ai in fluid communication with the first barrel outlet 1014a, a second mixing section inlet 1018bi in fluid communication with the second barrel outlet 1014b, and a mixing section outlet 10180. Also shown are a syringe holder 1022 configured to hold the first and second syringe barrels 1012a, 1012b, in a fixed relationship and a plunger cap 1024 configured to hold the first and second plungers 1019a, 1019b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a hydrolysis-resistant crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of system that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

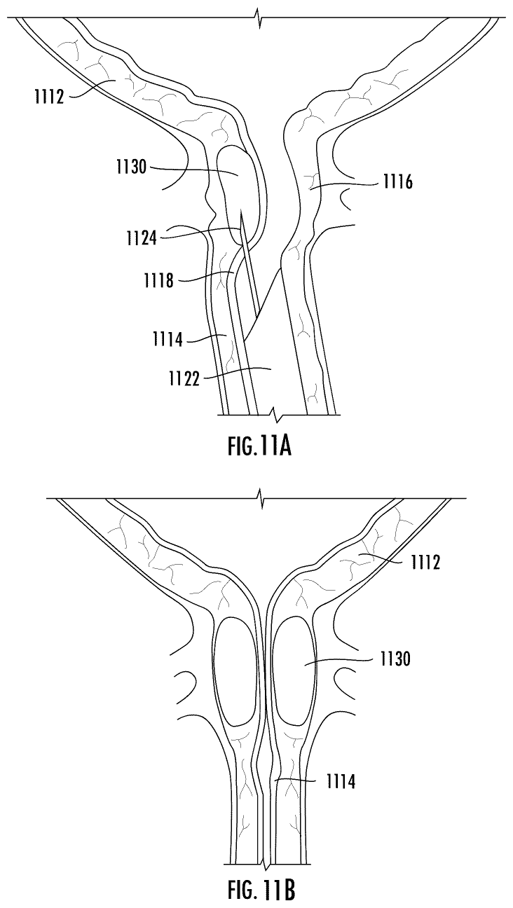

For example, in some embodiments, the first and second fluid compositions or a fluid admixture thereof can be injected as a bulking agent. For example, the first and second fluid compositions or a fluid admixture thereof may be injected to increase coaptation of a bodily sphincter, such as an anal sphincter for fecal incontinence or a urinary sphincter. FIGS. 11A and 11B illustrate a portion of the human anatomy that includes a lower portion of the bladder wall 1112, an upper portion of the urethra 1114, and the bladder neck 1116, which a group of muscles that connect the bladder to the urethra. A fluid admixture of the first and second fluid compositions is injected by a transurethral route using a suitable delivery device such as that shown in FIG. 10, where a catheter tube (not shown) and needle 1124 are attached to the mixing section outlet 10180 of the delivery device 1010. In the embodiment shown, the injection is performed with the aid of a cystoscope 1122 through the urethral wall 1118 into the bladder neck 1116, increasing coaptation of the urinary sphincter. After injection, the polyamino compound and the reactive polymer in the fluid admixture crosslink to form a hydrolysis-resistant hydrogel 1130.

In other examples, the first and second fluid compositions or a fluid admixture thereof can be injected as a bulking agent into tissue around the ureteral orifices for the treatment of vesicoureteral reflux, the first and second fluid compositions or a fluid admixture thereof can be injected for tissue augmentation or regeneration, including cosmetic tissue augmentation, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a hydrolysis-resistant crosslinked hydrogel is ultimately formed at the administration location.

During and/or after administration, the compositions of the present disclosure can be imaged using a suitable imaging technique. Typically, the imaging techniques is an x-ray-based imaging technique, such as computerized tomography or x-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to occlude either a vas deferens or fallopian tube with a crosslinked product of the first and second fluid compositions for the control of reproductive health/family planning, a procedure to implant an embolic composition comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, injection for obstruction of the vas deferens, injection for obstruction of the fallopian tube, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

In some embodiments, the hydrolysis-resistant crosslinked hydrogels are formed ex vivo, in which case the hydrolysis-resistant crosslinked hydrogels may be in any desired form, including a slab, a cylinder, a coating, or a particle. In some embodiments, the hydrolysis-resistant crosslinked hydrogel is dried and then granulated into particles of suitable size. Granulating may be by any suitable process, for instance by grinding (including cryogrinding), homogenization, crushing, milling, pounding, pressing through a screen, or the like. Sieving or other known techniques can be used to classify and fractionate the particles. Hydrogel particles formed using the above and other techniques may varying widely in size, for example, having an average size ranging from 50 to 950 microns.

In addition to a hydrolysis-resistant crosslinked hydrogel that is formed ex vivo, hydrogel compositions in accordance with the present disclosure may be provided, which contain additional agents, including therapeutic agents, imaging agents, colorants, tonicity adjusting agents, suspension agents, wetting agents, and pH adjusting agents as described above.

In various embodiments, kits are provided that include one or more delivery devices for delivering such hydrogel compositions to a subject. Such systems may include one or more of the following: a syringe barrel, which may or may not contain a hydrogel composition; a vial, which may or may not contain a hydrogel composition; a needle; a flexible tube (e.g., adapted to fluidly connect the needle to the syringe); and an injectable liquid such as water for injection, normal saline or phosphate buffered saline. Whether supplied in a syringe, vial, or other reservoir, the hydrogel composition may be provided in dry form (e.g., powder form) or in a form that is ready for injection, such as an injectable hydrogel form (e.g., a suspension of hydrogel particles).

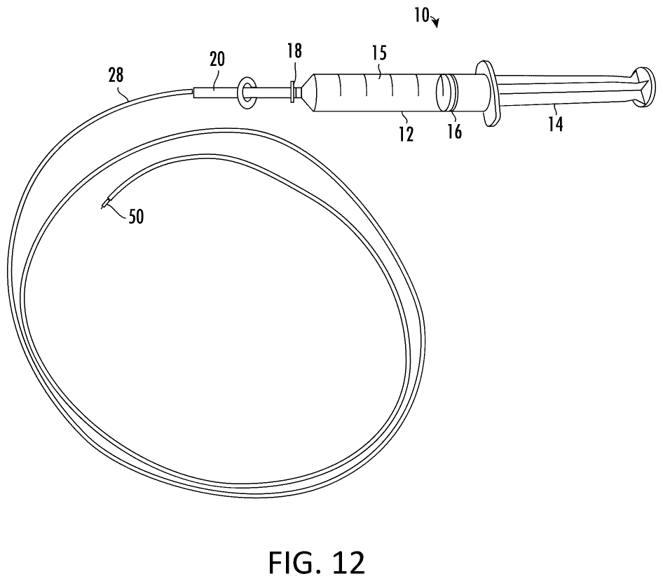

FIG. 12 illustrates a syringe 10 providing a reservoir for a hydrogel composition in accordance with the present disclosure. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a hydrogel composition 15 for injection through the needle 50.

The hydrogel compositions described herein (e.g., a suspension of hydrogel particles, which may also optionally contain additional agents described above) can be used for a number of purposes.

For example, hydrogel compositions can be injected to provide spacing between tissues, hydrogel compositions can be injected (e.g., in the form of blebs) to provide fiducial markers, hydrogel compositions can be injected for tissue augmentation or regeneration, hydrogel compositions can be injected as a filler or replacement for soft tissue, hydrogel compositions can be injected for tissue bulking, hydrogel compositions can be injected for occlusion of lumens, hydrogel compositions can be injected to provide mechanical support for compromised tissue, hydrogel compositions be injected as a scaffold, and/or hydrogel compositions can be injected as a carrier of therapeutic agents in the treatment of diseases and cancers and the repair and regeneration of tissue, among other uses.

During or after administration, the hydrogel compositions of the present disclosure can be imaged using a suitable imaging technique.

As seen from the above, the hydrogel compositions of the present disclosure may be used in a variety of medical procedures, including the following, among others: a procedure to implant a fiducial marker comprising a hydrogel, a procedure to implant a tissue regeneration scaffold comprising a hydrogel, a procedure to implant a tissue support comprising a hydrogel, a procedure to implant a tissue bulking agent comprising a hydrogel, a procedure to occlude a lumen, a procedure to implant a therapeutic-agent-containing depot comprising a hydrogel, a tissue augmentation procedure comprising implanting a hydrogel, a procedure to introduce a hydrogel between a first tissue and a second tissue to space the first tissue from the second tissue, among others.

The hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection to increase coaptation of a bodily sphincter such as an anal sphincter for or a urinary sphincter, injection for vas deferens occlusion, injection for fallopian tube occlusion, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Hydrogel compositions in accordance with the present disclosure include lubricious compositions for medical applications, compositions for therapeutic agent release (e.g., by including one or more therapeutic agents in a matrix of the hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. A system for forming a hydrogel that comprises (i) a reactive multi-arm polymer that comprises three or more polymer arms linked to a core region, at least three of the polymer arms comprising a hydrophilic polymer segment and a reactive moiety comprising a cyclic-imidyl-oxycarbonyl-C₂-C₈-alkylene-carbonylamino- end group and (ii) a polyamino compound comprising at least two amino (—NH₂) groups, wherein the reactive multi-arm polymer and the polyamino compound react to form a crosslinked hydrogel that does not contain ester groups and has long-term stability in vivo.

2. The system of claim 1, wherein the hydrophilic polymer segment is selected from polyalkylene oxide segments, polyester segments, polyoxazoline segments, polydioxanone segments, polypeptide segments, polyacrylamide segments, and poly(2-hydroxyethyl methacrylate) (PolyHEMA) segments.

3. The system of claim 1, wherein the reactive moiety comprises a cyclic-imidyl-oxycarbonyl-1,2-dimethylene-carbonylamino- end group, a cyclic-imidyl-oxycarbonyl-1,3-trimethylene-carbonylamino- end group, a cyclic-imidyl-oxycarbonyl-1,4-tetramethylene-carbonylamino- end group, a cyclic-imidyl-oxycarbonyl-1,5-pentamethylene-carbonylamino- end group, or a cyclic-imidyl-oxycarbonyl-1,6-hexamethylene-carbonylamino- end group.

4. The system of claim 1, wherein the reactive multi-arm polymer further comprises at least one iodinated aromatic group.

5. The system of claim 1, wherein the reactive multi-arm polymer is formed by a process that comprises (a) reacting a multi-arm polymer that comprises three or more precursor polymer arms linked to the core region, at least three of the precursor polymer arms comprising the hydrophilic polymer segment and an amino end group, with a cyclic anhydride having a ring that comprises the —C₂-C₈-alkylene- group, and (b) reacting the reaction product of step (a) with an N-hydroxy cyclic imide compound in an ester coupling reaction to form the cyclic-imidyl-oxycarbonyl-C₂-C₈-alkylene-carbonylamino- end group.

6. The system of claim 1, wherein the core region comprises a polyol residue.

7. The system of claim 1, wherein the polyamino compound comprises a plurality of basic amino acid residues.

8. The system of claim 1, wherein the polyamino compound is an iodinated polyamino compound.

9. The system of claim 1, comprising a first composition that comprises the polyamino compound in a first container and a second composition that comprises the reactive multi-arm polymer in a second container.

10. A hydrolysis-resistant crosslinked hydrogel produced by covalent crosslinking between (i) a reactive multi-arm polymer that comprises three or more polymer arms linked to a core region, at least three of the polymer arms comprising a hydrophilic polymer segment and a reactive moiety comprising a cyclic-imidyl-oxycarbonyl-C₂-C₈-alkylene-carbonylamino- end group and (ii) a polyamino compound comprising at least two amino (—NH₂) groups.

11. The hydrolysis-resistant crosslinked hydrogel of claim 10, wherein the hydrolysis-resistant crosslinked hydrogel has a radiopacity that is greater than 100 Hounsfield units (HU).

12. A method of treatment comprising administering to a subject a mixture that comprises the reactive multi-arm polymer and the polyamino compound of claim 1, wherein the mixture is administered under conditions such that the polyamino compound and the reactive multi-arm polymer react to form a crosslinked hydrogel after administration.

13. The method of claim 12, wherein the method comprises administering to the subject (a) a first fluid composition that comprises the polyamino compound and the reactive multi-arm polymer, and (b) a second fluid composition that comprises an accelerant that accelerates reaction of the polyamino compound and the reactive multi-arm polymer.

14. A system for forming a hydrogel that comprises (i) a reactive multi-arm polymer that comprises three or more polymer arms linked to a core region, at least three of the polymer arms comprising a polyoxazoline segment and a reactive moiety comprising a cyclic-imidyl-oxycarbonyl- end group and (ii) a polyamino compound comprising at least two amino (—NH₂) groups, wherein the reactive multi-arm polymer and the polyamino compound react to form a crosslinked hydrogel that does not contain ester groups and has long-term stability in vivo.

15. The system of claim 14, wherein the polyoxazoline is a poly(2-C₁-C₆-alkyl-2-oxazoline) and wherein the reactive moiety comprises a cyclic-imidyl-oxycarbonyl-N—C₂-C₇-alkanoyl-methylamino- end group.

16. The system of claim 14, wherein the polyoxazoline segment is selected from a poly(2-methyl-2-oxazoline) segment, a poly(2-ethyl-2-oxazoline) segment, a poly(2-n-propyl-2-oxazoline) segment, a poly(2-isopropyl-2-oxazoline) segment, a poly(2-n-butyl-2-oxazoline) segment, and a poly(2-isobutyl-2-oxazoline) segment.

17. The system of claim 14, wherein the reactive multi-arm polymer further comprises at least one iodinated aromatic group.

18. The system of claim 14, wherein the core region comprises at least one iodinated aromatic group.

19. The system of claim 14, wherein the reactive multi-arm polymer further comprises at least one polymer arm that comprises at least one iodinated aromatic end group.

20. The system of claim 14, wherein the reactive multi-arm polymer is formed by a process that comprises (a) reacting a multi-arm polymer that comprises three or more precursor polymer arms linked to the core region, at least three of the precursor polymer arms comprising the polyoxazoline segment and a hydroxyethyl end group, with an oxidizing agent that converts the hydroxyethyl end group to a carboxymethyl end group, and (b) reacting the reaction product of step (a) with an N-hydroxy cyclic imide compound in an ester coupling reaction to form the cyclic-imidyl-oxycarbonyl- end group.
